(19) United States
(12) Patent Application Publication
(10) Pub. No.: US 2004/0091947 A1
(43) Pub. Date: May 13, 2004

(54) Screening strategy for anticancer drugs

(76) Inventors: Eugenia Broude, Cohoes, NY (US); Igor B. Roninson, Cohoes, NY (US); Mari E. Swift, Kirkland, WA (US)

Correspondence Address: McDonnell Boehnen Hulbert & Berghoff, 32nd Floor, 300 S. Wacker Drive, Chicago, IL, 60606, US

(21) Appl. No.: 10/639,977

(22) Filed: Aug. 13, 2003

Related U.S. Application Data
(60) Provisional application No. 60/402,995, filed on Aug. 13, 2002.
(60) Provisional application No. 60/477,465, filed on Jun. 10, 2003.

(51) Int. Cl. G01N 33/574

(52) U.S. Cl. 435/7.23

(57) ABSTRACT

The invention provides methods and reagents for identifying compounds that growth inhibit or kill tumor cells.

This application was supported by a grant from the National Institutes of Health, No. R01 CA95727. The government may have certain rights in this invention.

### BACKGROUND OF THE INVENTION

This application claims priority to U.S. Provisional Application Serial Nos. 60/402,995, filed Aug. 13, 2002 and 60/477,465, filed Jun. 10, 2003

1. Field of the Invention

The invention is related to methods and reagents for inhibiting tumor cell growth. Specifically, the invention provides methods for identifying compounds, such as chemotherapeutic drugs, that permanently growth inhibit or kill tumor cells. The methods of the invention identify such drugs by assaying cellular responses to incubating cells in the presence of such drugs, wherein compounds that produce senescence or mitotic catastrophe in the cells are identified. Methods for using such drugs for treating tumor-bearing animals including humans are also provided.

2. Summary of the Related Art

Therapeutic efficacy of anticancer agents is determined by their ability to interfere with the growth or survival of tumor cells preferentially to normal cells. As reviewed in Roninson et al. (2001, Drug Resist. Updat. 4: 303-313), the antiproliferative effects of anticancer agents with proven clinical utility, including chemotherapeutic drugs and ionizing radiation, are mediated by three documented cellular responses. These responses include programmed cell death (apoptosis), abnormal mitosis that results in cell death (mitotic catastrophe), and permanent cell growth arrest (senescence). The first two responses result in the destruction and disappearance of tumor cells, whereas senescence prevents further cell proliferation but leaves tumor cells viable and metabolically active. As reviewed in Roninson (2003, Cancer Res. 63: 2705-2715), senescent tumor cells may produce two types of secreted proteins, some of which stimulate and others inhibit the growth of non-senescent neighboring tumor cells. In some cases, senescent tumor cells overproduce secreted growth-inhibitory proteins preferentially to tumor-promoting proteins, thereby rendering senescent cells that are a permanent reservoir of tumor-suppressive factors that assist in stopping tumor growth (Roninson, 2003, Id.).

Two of the antiproliferative responses, apoptosis and senescence, represent physiological anti-carcinogenic programs that are extant in normal cells. These programs are activated, among other factors, by oncogenic mutations, such as increased expression of C-MYC (that promotes apoptosis) or RAS mutations (that trigger senescence). However, during the course of carcinogenesis, tumor cells develop various genetic and epigenetic changes that suppress the apoptosis or senescence programs; these changes include mutational inactivation of p53 (which serves as a positive regulator of both apoptosis and senescence) or p16^{Ink4a} (a mediator of senescence), and upregulation of BCL-2 (an inhibitor of apoptosis). Despite these carcinogenesis-associated changes, it is still possible to induce apoptosis or senescence in tumor cells by treatment with certain anticancer agents. However, the efficacy of apoptosis and senescence for growth inhibiting tumor cells varies greatly among tumor-derived cell lines (Chang et al., 1999, Cancer Res. 59: 3761-3767; Roninson et al., 2001, Id.).

Analysis of the importance of apoptosis in treatment response is complicated by the fact that apoptosis frequently develops not as a primary effect of cellular damage but as a secondary response consequent to abnormal mitosis (Roninson, 2001, Drug Resist. Updat. 5: 204-208). Without apoptosis, abnormal mitosis ends in micronucleation (i.e., formation of large interphase cells with completely or partially fragmented nuclei). Both post-mitotic apoptosis and micronucleation can be viewed as alternative lethal outcomes of mitotic catastrophe. Lock and Stribinskiene (1996, Cancer Res. 56: 4006-4012) and Ruth and Roninson (2000, Cancer Res. 60: 2576-2578) found that inhibition of the apoptotic program in drug-treated or irradiated cells resulted in an increase in the fraction of cells that die through micronucleation (the latter study also showed concurrent increase in the fraction of senescent cells). As a consequence, apoptosis inhibition in many human tumor cell lines was found to have little or no effect on the ability of drug-treated or irradiated cells to proliferate (Borst et al., 2001, Drug Resist. Update 4: 128-130; Roninson et al., 2001, Id.).

In contrast to apoptosis or senescence, mitotic catastrophe does not represent a normal physiological program but instead results from entry of damaged cells into mitosis under suboptimal conditions. Normal cells possess a variety of cell cycle checkpoint mechanisms that prevent inauspicious entry into mitosis, e.g., after chromosomal DNA has been damaged but before repair mechanisms can restore the damaged DNA. These include, among others, DNA damage-inducible checkpoints that arrest cells in either G1 or G2 phases of the cell cycle, or the prophase checkpoint activated by microtubule-targeting drugs. Checkpoint arrest gives cells time to repair cellular damage, particularly chromosomal DNA damage, and reduces the danger of abnormal mitosis.

Tumor cells, on the other hand, are almost always deficient in one or more of these cell cycle checkpoints, and exploiting these deficiencies is a major direction in experimental therapeutics (O'Connor, 1997, Cancer Surv. 29: 151-182; Pihan and Doxsey, 1999, Semin. Cancer Biol. 9: 289-302). For example, tumor cells frequently inactivate the tumor suppressor p53 required for the G1 checkpoint, as well as such genes as ATM or ATR that mediate the G2 checkpoint, and the CHFR gene that mediates the prophase checkpoint in non-tumor cells. Scolnick and Halazonetis (2000, Nature 406: 430-435) disclosed that a high fraction of tumor cell lines are deficient in CHFR. In the presence of antimicrotubular drugs, CHFR appears to arrest the cell cycle at prophase. CHFR-deficient tumor cells, however, proceed into drug-impacted abnormal metaphase (Scolnick and Halazonetis, 2000, Id.), where they die through mitotic catastrophe or apoptosis (Torres and Horwitz, 1998, Cancer Res. 58: 3620-3626). Inactivation of these checkpoints has been shown to promote mitotic catastrophe after treatment with anticancer drugs or radiation (Roninson et al., 2001, Id.).

The role of mitotic catastrophe as a principal tumor-specific antiproliferative response to clinically useful anticancer agents has been neither suggested nor experimentally tested in the prior art. Most studies where mitotic catastrophe was induced in tumor cells preferentially to normal cells involved situations where tumor cells preferentially entered mitosis, and such studies did not investigate whether the ratio of normal and abnormal mitoses differed between similarly treated tumor and normal cells. For example, Powell et al. (1995, Cancer Res. 55: 1643-1648) showed that caffeine, an agent that abrogates damage-induced G2 checkpoint, sensitizes mammalian cells to radiation-induced cell death, and that this sensitization was specific for cells lacking functional p53. More recently, Jha et al. (2002, Radiat. Res. 157: 26-31) showed that G2 checkpoint abrogation by caffeine occurs in tumor cells but not in normal human cells. As shown by Nghiem et al. (2001, Proc. Natl. Acad. Sci. USA 98: 9092-9097), G2 checkpoint abrogation sensitizes p53-deficient cells to different DNA-damaging agents specifically by promoting mitotic catastrophe, associated here with “premature chromosome condensation.” Similarly, Qiu et al. (2000, Molec. Biol. Cell 11: 2069-2083) reported that histone deacetylase inhibitors (HDAC-I) triggered a G2 checkpoint in normal human fibroblasts but not in tumor cell lines. Consequent to HDAC-I treatment, tumor cells entered abnormal mitosis and died through mitotic catastrophe, whereas HDAC-I treated normal cells became arrested in G2 and did not enter mitosis (Qiu et al., 2000, Id.).

In a different type of study, Cogswell et al. (2000, Cell Growth Differ. 11: 615-623) demonstrated that a dominant-negative mutant of Polo-like kinase 1 (PLK1), an enzyme that plays a key role in mitosis, induced mitotic catastrophe in human tumor cells preferentially to normal mammary epithelial cells. In these studies, Cogswell et al. compared the frequency of normal and abnormal mitoses among normal and tumor cells infected with an adenoviral vector carrying dominant-negative PLK1, and showed that this vector produced abnormal mitosis in tumor cells more frequently than in normal cells. Cogswell et al. suggested that this differential response of tumor and normal cells could potentially reflect a greater dependence of tumor cells (which overexpress PLK1) on PLK1 for formation of essential mitotic complexes. In other words, the tumor specificity of this response was considered to be specific for PLK1 inhibition.

All classes of anticancer drugs in today's clinical armamentarium induce both mitotic catastrophe and senescence (Chang et al., 1999, Id). None of these agents, however, have been discovered on the basis of their ability to induce these useful antiproliferative responses. Directed screening strategies for compounds that induce either mitotic catastrophe or senescence in tumor cells should be useful in finding agents with greater efficacy and tumor specificity than the presently available drugs. There are as yet no reports of drug screening based on the ability to induce senescence, but screening strategies based on the use of senescence-associated genes as markers are a subject of co-owned and co-pending patent applications (see, International Patent Applications, Publication Nos. WO01/92578 and WO02/061134). Rather, the prior art contains screening strategies for producing agents that induce mitotic arrest (Mayer et al., 1999, Science 286:971-974; Roberge et al., 2000, Cancer Res. 60: 5052-5058; Haggarty et al., 2000, Chem. Biol 7: 275-286). Other prior art approaches involve identifying compounds that override the G2 checkpoint, thus permitting cells with damaged DNA to enter mitosis before repairing the damage (Roberge et al., 1998, Cancer Res 58: 5701-5706); however, such agents do not directly induce but rather promote mitotic catastrophe in cells treated with DNA-damaging drugs.

Agents that affect mitosis or cellular entry into mitosis, however, are not the only ones that can induce mitotic catastrophe. For example, all anticancer drugs that inhibit the cell cycle at interphase efficiently induce mitotic catastrophe (Chang et al., 1999, Id.). Furthermore, tumor cells reentering the cycle after cytostatic growth inhibition by a cyclin-dependent kinase inhibitor p21^{Waf1/Cip1/Sdi1} also undergo catastrophe upon entering mitosis (Chang et al., 2000, Oncogene 19: 2165-2170).

There remains a need in the art to identify compounds that exploit cancer-related phenotypic differences between tumor cells and normal cells from the tissues in which tumors arise, as a way to preferentially promote cell death in tumor rather than normal cells. There also remains a need in the art to identify compounds that induce senescence in tumor cells and thereby stop tumor growth.

### SUMMARY OF THE INVENTION

The invention provides methods for identifying compounds that permanently inhibit cell growth or kill tumor cells.

In a first aspect, the invention provides methods for identifying compounds that induce cell death in tumor cells preferentially to normal cells. As shown herein, a commonly used anticancer drug preferentially induces mitotic catastrophe (rather than senescence or apoptosis) in neoplastically-transformed cells relative to isogenic normal cells. Hence, agents that induce mitotic catastrophe in tumor cells are likely to act in a tumor-specific manner. In certain embodiments, the methods of the invention comprise the steps of a) contacting a cancer cell culture with a test compound, with or without subsequent removal of the compound; and b) assaying compounds for induction of mitotic catastrophe, by assessing the morphology of mitotic figures in the treated cells or by detecting the appearance in the culture of interphase cells having two or more micronuclei. In additional aspects, the invention provides methods for verifying tumor-specific cytotoxicity of the identified compounds. These aspects of the methods of the invention comprise the additional steps of contacting a culture of non-cancer cells with the compound for a time and at a compound concentration sufficient to induce mitotic catastrophe in tumor cells; assaying compounds for the induction of cell death; and identifying compounds that do not induce or only weakly induce cell death in non-cancer cells.

In a second aspect, the invention provides efficient screening methods for identifying cytostatic agents that induce either mitotic catastrophe or senescence in tumor cells. In certain embodiments, the methods of the invention comprise the steps of a) contacting a cancer cell culture with a test compound for a time and at a compound concentration sufficient to induce cell growth arrest in the cells; b) assaying a portion of the treated cells to detect a decrease in the mitotic index of the treated cells; c) removing the compound and culturing the cells for a recovery period comprising a time sufficient to permit the cells to re-enter the cell cycle; d) assaying a portion of the recovered cells to detect an increase in the mitotic index of the recovered cells; e) assaying compounds producing an increase in mitotic index smaller than in untreated cells for induction of senescence, by detecting production of senescence markers in said cells; f) assaying compounds producing increases in mitotic index as large or larger than in untreated cells for mitotic catastrophe, by assessing mitotic figure morphology in the treated and recovered cells or by detecting the appearance in the culture of interphase cells with two or more micronuclei; and g) identifying compounds that induce small increases in mitotic index and expression of senescence markers as senescence-inducing compounds in cancer cells, and identifying compounds that induce abnormal mitotic figures or micronuclei in the cells as compounds that induce mitotic catastrophe in cancer cells. In preferred embodiments, the cells are human cancer cells, more preferably solid tumor cells and most preferably HT1080 cells. In additional preferred embodiments, the cells are assayed in step (b) to detect a decrease in the mitotic index by staining a portion of the treated cells with a mitosis-specific reagent. Preferably, the mitosis-specific reagent is a mitotic cell-specific antibody. In certain embodiments, the cells are assayed by microscopy or by florescence-activated cell sorting. In additional embodiments, the cells are assayed in step (d) to detect an increase in the mitotic index by staining a portion of the recovered cells with a mitosis-specific reagent. Preferably, the mitosis-specific reagent is a mitotic cell-specific antibody. In certain embodiments, the cells are assayed by microscopy or by fluorescence-activated cell sorting. After incubation and release according to the methods of this aspect of the invention, cells showing a small increase in mitotic index are assayed with a senescence marker that is senescence-associated beta-galactosidase (SA-β-gal) or tested for the ability to abrogate long-term colony formation. In cells showing a large increase in mitotic index, chromosomal morphology is advantageously assayed using a DNA-specific detection reagent and detected using microscopy or by fluorescence-activated cell sorting. Alternatively, chromosomal morphology is assayed using an H2B-GFP fusion protein.

In a third aspect, the invention provides methods for inhibiting tumor cell growth, the method comprising the steps of contacting a tumor cell with an effective amount of a compound that induces mitotic catastrophe in a cancer cell, identified according to the methods of the invention.

In a fourth aspect, the invention provides methods for treating a disease or condition relating to abnormal cell proliferation or tumor cell growth, the method comprising the steps of contacting a tumor cell with an effective amount of a compound that induces mitotic catastrophe in a cancer cell, identified according to the methods of the invention.

A fifth aspect of the invention provides compounds that inhibit tumor cell growth, wherein the compound that induces mitotic catastrophe in a cell is identified according to the methods of the invention.

In a sixth aspect, the invention provides methods for inducing senescence in a cancer cell. In these embodiments, the methods comprise the step of contacting a tumor cell with an effective amount of a compound that stably decreases the mitotic index when the cell is contacted with the compound.

In a seventh aspect, the invention provides methods for treating a disease or condition relating to abnormal cell proliferation or tumor cell growth, the method comprising the steps of contacting a tumor cell with an effective amount of a compound that stably decreases the mitotic index when the cell is contacted with the compound.

In an eighth aspect, the invention provides compounds that induce senescence in a cancer cell, wherein the compound stably decreases the mitotic index when the cell is contacted with the compound.

Pharmaceutically acceptable compositions effective according to the methods of the invention, comprising a therapeutically effective amount of a peptide or peptide mimetic of the invention capable of inhibiting tumor cell growth and a pharmaceutically acceptable carrier or diluent, are also provided.

Specific preferred embodiments of the present invention will become evident from the following more detailed description of certain preferred embodiments and the claims.

### BRIEF DESCRIPTION OF THE DRAWINGS

FIG. 1 is a schematic diagram illustrating the screening strategy employed according to the methods of the invention.

FIG. 2 shows photomicrographs of fluorescently-stained chromosomes showing abnormal mitotic figures characteristic of mitotic catastrophe.

FIGS. 3A through 3E are graphs showing the number of cells/well for untreated (FIG. 3A) and doxorubicin-treated (FIG. 3B) BJ-EN and BJ-ELB cells, and the number of cells/well (FIG. 3C), percent SA-β-gal expressing (FIG. 3D) and percent mitotic index (FIG. 3E) for BJ-EN and BJ-ELB cells treated with doxorubicin and then incubated in media without doxorubicin for three days, as described in Example 1.

FIG. 4 are photomicrographs of fluorescently-stained chromosomes showing normal and abnormal mitotic figures produced in BJ-EN and BJ-ELB cells as set forth in Example 1.

FIGS. 5A and 5B are fluorescence activated cell sorting plots (FIG. 5A) showing the effects of radiation on MI: GF7 and PI staining of GSE56-transduced cells, untreated or analyzed 9 h after 9 Gy irradiation in the presence of caffeine; and (FIG. 5B) a plot of the time course of changes in GF7+ fraction in control and GSE56-transduced cells, after 9 Gy irradiation in the presence and in the absence of caffeine.

### DETAILED DESCRIPTION OF PREFERRED EMBODIMENTS

Clinically useful anticancer agents permanently stop the growth of tumor cells by inducing apoptosis (programmed cell death), mitotic catastrophe (cell death that results from abnormal mitosis), or senescence (permanent cell growth arrest). The properties and characteristics of these three processes are shown in Table 1.

This invention provides cell-based screening strategies that can identify compounds that induce mitotic catastrophe or senescence in a cell, preferably a tumor cell and most preferably a tumor cell rather than a normal cell from a tissue in which the tumor cell arose. These strategies can be used for more efficient screening of natural and synthetic compound libraries for agents with anticancer activity.

Standard techniques may be used in the practice of the methods of this invention for tissue culture, drug treatment and transformation (e.g., electroporation, lipofection). The foregoing techniques and procedures may be generally performed according to conventional methods well known in the art and as described in various general and more specific references that are cited and discussed throughout the present specification. See e.g., Sambrook et al., 2001, MOLECULAR CLONING: A LABORATORY MANUAL, 3d ed., Cold Spring Harbor Laboratory Press, Cold Spring Harbor, N.Y., which is incorporated herein by reference for any purpose, and Freshney, 2000, CULTURE OF ANIMAL CELLS: A MANUAL OF BASIC TECHNIQUE, Wiley-Liss: New York, which is incorporated herein by reference for any purpose. Unless specific definitions are provided, the nomenclature utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical

TABLE 1
Anti proliferative effects of anticancer agents

| | Apoptosis | Mitotic catastrophe | Senescence |
|---|---|---|---|
| Definition | Programmed cell death. | Cell death that results from abnormal | Programmed terminal growth |
| | | mitosis. | arrest. |
| Inducing agents | All chemotherapeutic drugs, | All chemotherapeutic drugs, radiation, | All DNA-interactive drugs, |
| | radiation, inducers of | inhibitors of mitotic proteins (e.g. polo | radiation, differentiating |
| | apoptotic pathways (e.g. | kinase inhibitors). | agents. |
| | FAS, TRAIL). | | Weakly induced by anti- |
| | | | microtubular drugs. |
| Morphological and | Shrunken cytoplasm, | (a) During abnormal mitosis (transient): | Large and flat cells, increased |
| biochemical changes | fragmented nuclei, | condensed chromatin, no nuclear | granularity, staining for β- |
| | condensed chromatin, inter- | envelope, abnormal mitotic figures. | galactosidase activity at pH6.0 |
| | nucleosomal DNA | (b) After abnormal mitosis (stable): | (SA-β-gal). |
| | fragmentation, caspase | large cells, two or more micronuclei, | |
| | activation (in most cases). | uncondensed chromatin. | |
| | | Alternative endpoint: apoptosis. | |
| Effects of genetic | Stimulated by MYC or Cyclin | Stimulated by deficiencies in G1, G2 | Stimulated by Ras mutations |
| changes associated | D1 activation; inhibited by | and prophase checkpoint proteins | and telomere shortening; |
| with carcinogenesis | the loss of p53 or PTEN, | (such as p53, ATM, ATR, Chk2, inhibited by | the loss of p53 or |
| | activation of Bcl2, Bcl-xL and | Cdc25A, Cdc25B, Plk1, Prk, Mlh1, | p16, activation of telomerase. |
| | other antiapoptotic genes, | CHFR). | |
| | inactivation of Bax and other | | |
| | proapoptotic genes. | | |

analyses, pharmaceutical preparation, formulation, and delivery, and treatment of patients.

Standard techniques may be used in the practice of the methods of this invention for tissue culture, drug treatment and transformation (e.g., electroporation, lipofection). The foregoing techniques and procedures may be generally performed according to conventional methods well known in the art and as described in various general and more specific references that are cited and discussed throughout the present specification. See e.g., Sambrook et al., 2001, MOLECULAR CLONING: A LABORATORY MANUAL, 3d ed., Cold Spring Harbor Laboratory Press, Cold Spring Harbor, N.Y., which is incorporated herein by reference for any purpose, and Freshney, 2000, CULTURE OF ANIMAL CELLS: A MANUAL OF BASIC TECHNIQUE, Wiley-Liss: New York, which is incorporated herein by reference for any purpose. Unless specific definitions are provided, the nomenclature utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

For the purposes of this invention, reference to “a cell” or “cells” is intended to be equivalent, and particularly encompasses in vitro cultures of mammalian cells grown and maintained as known in the art.

For the purposes of this invention, the term “senescence” will be understood to include permanent cessation of DNA replication and cell growth not reversible by growth factors, such as occurs at the end of the proliferative lifespan of normal cells or in normal or tumor cells in response to cytotoxic drugs, DNA damage or other cellular insult. Senescence is also characterized by certain morphological features, including increased size, flattened morphology increased granularity, and senescence-associated β-galactosidase activity (SA-β-gal).

Senescence can be conveniently induced in mammalian cells by contacting the cells with a dose of a cytotoxic agent that inhibits cell growth (as disclosed in Chang et al., 1999, Id.). Cell growth is determined by comparing the number of cells cultured in the presence and absence of the agent and detecting growth inhibition when there are fewer cells in the presence of the agent than in the absence of the agent after an equivalent culture period of time. Examples of such cytotoxic agents include but are not limited to doxorubicin, aphidicolin, cisplatin, cytarabine, etoposide, taxol, ionizing radiation, retinoids or butyrates. Appropriate dosages will vary with different cell types; the determination of the dose that induces senescence is within the skill of one having ordinary skill in the art, as disclosed in Chang et al., 1999, Id.

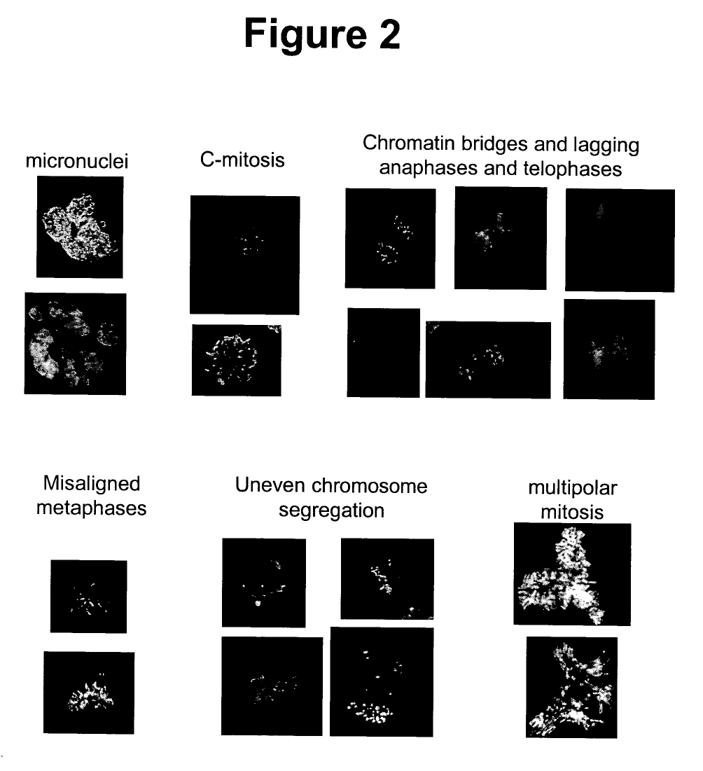

For the purposes of this invention, the term “mitotic catastrophe” will be understood to include any form of abnormal mitosis that results in cell death. Such cell death is frequently but not always preceded by the formation of micronucleated interphase cells, which are thus an indicator of mitotic catastrophe. In addition, mitotic catastrophe may also lead to apoptosis. Mitotic catastrophe can be conveniently induced in mammalian cells by contacting the cells with a cytotoxic agent (as disclosed in Chang et al., 1999, Id.). Mitotic catastrophe can be determined microscopically by observing mitotic figures that are clearly different from normal, as illustrated in FIG. 2, or by detecting interphase cells with two or more micronuclei, which may be completely or partially separated from each other. Examples of cytotoxic agents effective for inducing mitotic catastrophe include but are not limited to doxorubicin, aphidicolin, cisplatin, cytarabine, etoposide, ionizing radiation, taxol or Vinca alkaloids. Appropriate dosages will vary with different cell types; the determination of the dose that induces mitotic catastrophe is within the skill of one having ordinary skill in the art, as disclosed in Chang et al., 1999, Id.

For the purposes of this invention, the term “apoptosis” will be understood to include the process of programmed cell death characterized by shrunken cytoplasm, fragmented nuclei, and condensed chromatin (as reviewed in Trump et al., 1997, Toxicol. Pathol. 25: 82-88). Apoptosis may be induced directly by certain agents (such as FAS or TRAIL) or may occur in response to DNA damage or abnormal mitosis.

The prominence of these three responses in cell lines derived from human solid tumors (HT1080 cells, Accession No. CCL-121, American Type Culture Collection, Manassas, Va.) is disclosed in co-owned and co-pending U.S. Ser. No. 09/449,589, (filed Nov. 29, 1999, incorporated by reference herein). Treatment of HT1080 fibrosarcoma cells with ID85 doses of six DNA-damaging agents induced the senescent phenotype in 15-79% of the cells, but only 3-9% developed this response when treated with two anti-microtubular drugs. On the other hand, mitotic catastrophe was observed in 45-66% of the cells treated with any of the tested agents, but very few (<10%) HT1080 cells developed apoptosis after treatment with any of the drugs. This analysis was expanded to a panel of 14 solid tumor-derived cell lines that were treated with moderate equitoxic doses of doxorubicin. Only two lines showed predominantly apoptotic response, whereas all the other lines developed mitotic catastrophe, with or without apoptosis. Eleven of 14 lines also exhibited the senescent phenotype after doxorubicin treatment.

To analyze the relationship between apoptosis, mitotic catastrophe and accelerated senescence, Ruth and Roninson (2000, Id.) investigated the effect of the MDR1 P-glycoprotein (which inhibits apoptosis through a mechanism distinct from its well-known function as multidrug transporter), on radiation resistance. P-glycoprotein protected two apoptosis-prone cell lines from radiation-induced apoptosis, but it did not increase the clonogenic survival of radiation. This apparent paradox was resolved by finding that a decrease in the fraction of apoptotic cells was accompanied by a commensurate increase in the fraction of cells undergoing either senescence or mitotic catastrophe, indicating that the latter responses, without apoptosis, are sufficient to stop proliferation of tumor cells.

A great amount of effort over the past decade has been devoted to the identification of agents that induce or stimulate apoptosis in tumor cells, but there have been no comprehensive efforts to identify agents that induce senescence or mitotic catastrophe in cancer cells. The latter responses, however, are not only common in cancer treatment but also possess certain advantages over apoptosis as cancer treatment strategies. Cells that undergo senescence do not divide but remain metabolically and synthetically active and produce secreted factors with important paracrine activities. While some of these factors may promote tumor growth by inhibiting apoptosis or by acting as mitogens, other factors (such as maspin, IGF-binding proteins or amphiregulin) have the opposite, tumor-suppressive effect (as disclosed in co-owned and co-pending U.S. Ser. No. 09/861,925, filed May 21, 2001 and International Patent Application, Publication No. WO 02/066681, published Aug. 29, 2002, each incorporated by reference herein). Some inducers of senescence, such as retinoids, stimulate the production of tumor-suppressive but not of tumor-promoting proteins (as disclosed in co-owned and co-pending U.S. Ser. No. 09/865,879, filed May 25, 2002, incorporated by reference herein), turning senescent tumor cells into a reservoir of secreted factors that inhibit the growth of their non-senescent neighbors. In contrast to senescence, apoptotic cells rapidly die and disappear, and therefore do not produce any factors that may suppress the growth of tumor cells that had escaped lethal damage.

The advantages of mitotic catastrophe over apoptosis as a therapeutic endpoint for anticancer drug treatment are apparent from the following considerations. Apoptosis is a physiological anti-carcinogenic program of normal cells. In the course of carcinogenesis, tumor cells develop various changes that suppress apoptotic programs, such as mutational inactivation of p53 and upregulation of BCL-2 (an inhibitor of apoptosis). As a result, many tumor cells show diminished apoptotic response (as disclosed in co-owned and co-pending U.S. Ser. No. 10/032,264, filed Dec. 21, 2001, incorporated by reference herein). In contrast, mitotic catastrophe is not a physiological program but rather a consequence of direct interference with mitosis (the effect of anti-mitotic drugs, such as Vinca alkaloids or taxanes), or of the entry of cells, damaged at interphase, into mitosis. The latter situation occurs when cells treated with DNA-damaging agents or other drugs that act at interphase enter mitosis after exposure to the drug; abnormal mitosis can also occur after cell cycle perturbation without DNA damage, e.g. after release from growth arrest produced by cyclin-dependent kinase inhibitor p21^{Waf1/Cip1/Sdi1} (as disclosed in co-owned and co-pending U.S. Ser. No. 09/958,361, filed Oct. 11, 2000, incorporated by reference herein). Normal cells possess a variety of cell cycle checkpoint mechanisms that prevent the entry of damaged cells into mitosis. These include, among others, DNA damage-inducible checkpoints that arrest cells in either G1 or G2 phases of the cell cycle, and the prophase checkpoint activated by microtubule-targeting drugs. Checkpoint arrest gives cells time to repair cellular damage, particularly chromosomal DNA damage, and reduces the danger of abnormal mitosis. Tumor cells, however, are almost always deficient in one or more of the cell cycle checkpoints. For example, transformed cells frequently inactivate the tumor suppressor p53 required for the G1 checkpoint, as well as such genes as ATM or ATR that mediate the G2 checkpoint, and the CHFR gene that mediates the prophase checkpoint (Stewart and Pietenpol, 2001, “G2 checkpoints and anticancer therapy,” in CELL CYCLE CHECKPOINTS AND CANCER, (Blagosklonny, ed.), Georgetown, Tex.: Landes Bioscience, pp. 155-178; Scolnick and Halazonetis, 2000, Nature 406: 430-435). Inactivation of these checkpoints promotes mitotic catastrophe after treatment with anticancer drugs or radiation. Other advantages of mitotic catastrophe in clinical situations are that (i) mitotic catastrophe occurs at lower drug doses (and therefore under the conditions of lower systemic toxicity) than apoptosis (Tounekti et al., 1993, Cancer Res. 53: 5462-5469; Torres and Horwitz, 1998, Cancer Res. 58: 3620-3626), and (ii) cells and tumors undergoing mitotic catastrophe die primarily through necrosis involving local inflammation (Cohen-Jonathan et al., 1999, Curr. Opin. Chem. Biol. 3: 77-83), which may further assist in the eradication of the residual tumor (in contrast, the process of apoptosis is non-inflammatory).

As disclosed herein in Example 1, doxorubicin, a commonly used anticancer agent that arrests the cell cycle in late S and G2 phases, has differential effects on normal human BJ-EN fibroblasts immortalized by transduction with telomerase (hTERT) and their isogenic, partially transformed derivative BJ-ELB, transduced with both hTERT and the early region of SV40. The ability of doxorubicin to induce senescence, apoptosis and mitotic catastrophe was compared between BJ-EN and BJ-ELB lines. Doxorubicin induced senescence to a similar extent in both cell lines and showed relatively weak induction of apoptosis. This drug, however, produced mitotic catastrophe much more efficiently in partially transformed BJ-ELB than in normal BJ-EN cells, and this difference went along with the overall stronger inhibitory effect of doxorubicin on BJ-ELB than on BJ-EN cells. This finding demonstrates that mitotic catastrophe, rather than senescence or apoptosis is the key determinant of tumor specificity of this important, clinically-useful anticancer drug. This finding, together with the above-discussed role of checkpoint deficiencies of tumor cells in promoting mitotic catastrophe, demonstrates that mitotic catastrophe is a tumor-specific mechanism of cell death. Hence, compounds that induce mitotic catastrophe in cancer cells are likely to have a tumor-specific effect, that is, to induce mitotic catastrophe and cell death in cancer cells but not in non-cancer cells. Such compounds can be identified by microscopic assays for abnormal mitotic figures or interphase cells having two or more micronuclei, a common endpoint of mitotic catastrophe. The tumor specificity of such compounds can then be verified by determining that the compounds do not induce or only weakly induce cell death in non-cancer cells. Cell death can be monitored by any standard procedure, such as detecting the appearance of apoptotic cells, or interphase cells with two or more micronuclei, or floating cells, or cells permeable to a dye that does not penetrate live cells (such as trypan blue).

The instant invention also provides efficient screening methods for compounds that induce either mitotic catastrophe or senescence. Screening synthetic or natural compound libraries for agents that induce mitotic catastrophe or senescence is based on measuring the fraction of mitotic cells (mitotic index, MI) in a cell culture after treatment with a tested compound. MI measurement has been previously used as the basis of screening for drugs that induce mitotic arrest. Such anti-mitotic drugs slow down or block mitosis, resulting in a strong increase in MI. Increased MI has been used in the art to screen for novel anti-mitotic drugs (Mayer et al., 1999, Science 286:971-974; Roberge et al., 2000, Cancer Res. 60: 5052-5058; Haggarty et al., 2000, Chem. Biol 7: 275-286). Another type of mitosis-based screening assays is aimed at identifying agents (such as caffeine or UCN-01) that override the G2 checkpoint; such agents can be identified by their ability to prevent the decrease in MI of nocodazole-treated cells after the infliction of DNA damage (Roberge et al., 1998, Cancer Res 58: 5701-5706).

MI-based assays known in the prior art, however, cannot detect cytostatic agents that induce mitotic catastrophe after arresting the cell cycle at interphase rather than acting directly at mitosis (such as DNA-damaging drugs), or cytostatic agents that induce senescence, which is associated with permanent growth arrest in G1 or G2. Both classes of the latter agents induce cell cycle arrest in the interphase rather than at mitosis and therefore decrease rather than increase the MI. The measurement of MI in the presence of such agents can therefore be used as the first step of screening for both classes of agents. An increase in MI will indicate potential anti-mitotic drugs (as in previously described assays), whereas a decrease in MI provides a novel criterion for identifying interphase-acting cell cycle inhibiting agents.

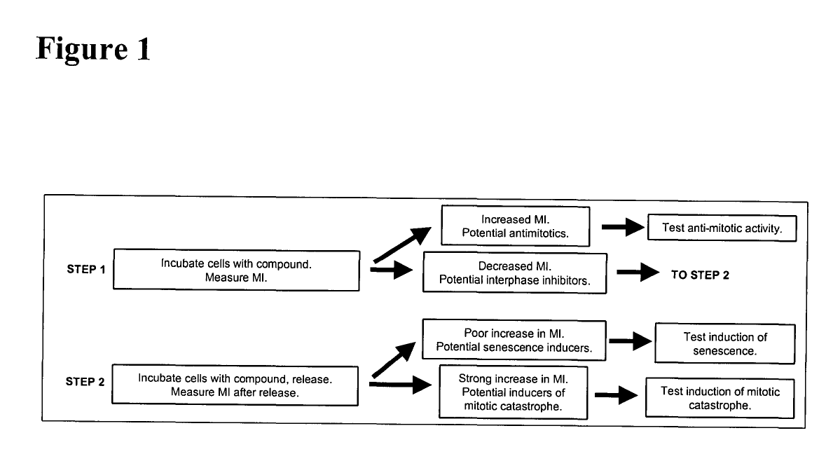

Agents inducing senescence or mitotic catastrophe can be distinguished by monitoring changes in MI after release from culture in the presence of the compound. Senescence-inducing agents will not permit full recovery of MI after release from the compound. In contrast, agents that induce mitotic catastrophe will not only permit recovery of MI but are likely to produce an increase in MI relative to control cells, since abnormal mitosis is expected to take longer than normal mitosis. For example, Mikhailov et al. (2002, Curr Biol 12: 1797-1806) showed that DNA damage during prophase delays exit from mitosis due to defects in kinetochore attachment and function. The extent of MI recovery after release from the compound will therefore identify compounds that induce either senescence or mitotic catastrophe. The effects of such compounds can then be verified by conventional assays for these two responses (as set forth in Table 1). This screening strategy is schematically illustrated in FIG. 1.

As shown in FIG. 1, the screening methods of the invention generally comprise two steps. In the first step, tumor cells are incubated in the presence of a test compound and the mitotic index (MI) measured. The time of incubation should be long enough to produce a significant change in the fraction of cells entering mitosis; it may be as short as 2-3 hours (a typical duration of the G2 phase) or as long as the duration of the entire cell cycle (between 20 hr and 45 hr for most tumor cell lines) or longer.

The informative consequences of incubation in the presence of a test compound are that MI either increases or decreases. Compounds showing increased MI are identified as potential antimitotic agents, which can then be tested for antimitotic activity using methods well known in the art. Compounds in whose presence cells show decreased MI are identified as interphase-acting cell cycle inhibitors and are used in the second step of the assay.

In the second step, cells are contacted with an effective amount of the test compound that causes a decrease in MI in step 1, for a time sufficient for decreased MI to be detected. Typically, this amount of time is also identified in step 1 of the inventive methods. Thereafter, the cells are released from test compound treatment, for example, by growth in culture media lacking the test compound. The length of time for test compound-free cell growth should be sufficient to allow the cells to re-enter the cycle, and is typically permitted from between 1 and 5 days. The MI of the cells during this time is determined.

One informative consequence of this treatment is a poor (i.e., small) increase in MI, for example, where the MI value does not reach the level observed in untreated cells grown to the same density. This result suggests that some of the treated cells have become stably growth-arrested, which is likely to reflect that they have become senescent. The induction of senescence by the compound can be experimentally determined, inter alia, by assaying the cells for senescence markers such as senescence-associated beta-galactosidase (SA-β-gal) expression, or for the expression of senescence-associated genes, as disclosed in co-owned and co-pending International Patent Application, Publication No., WO02/061134.

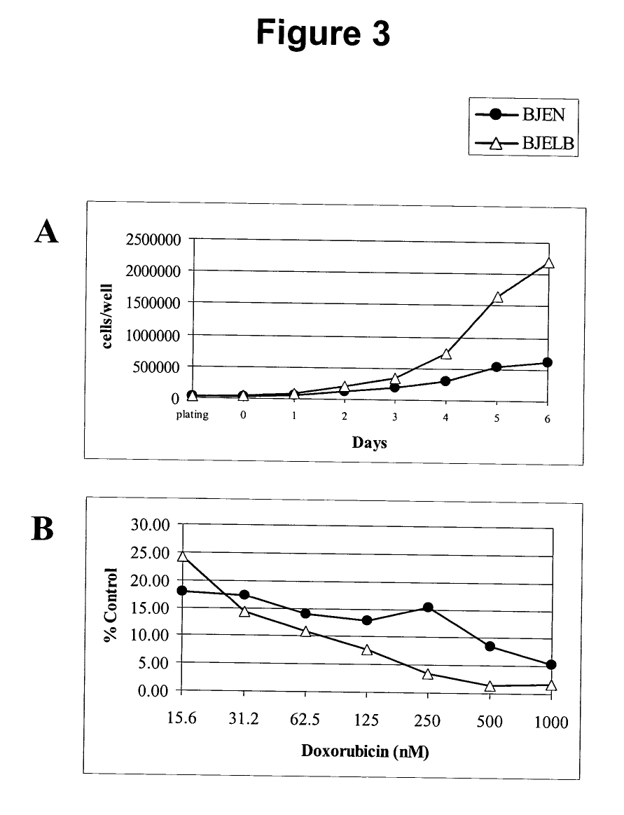

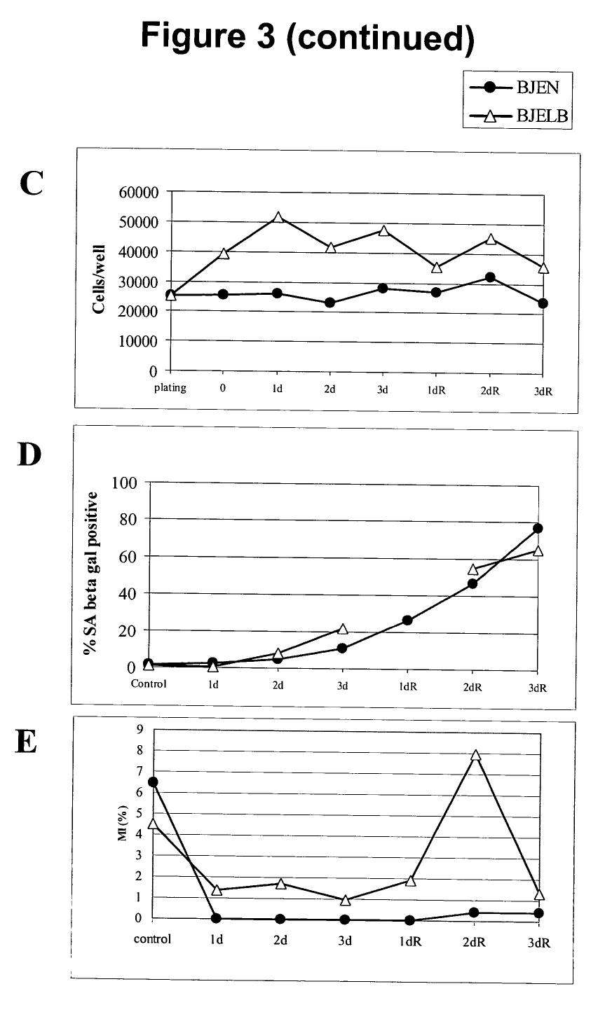

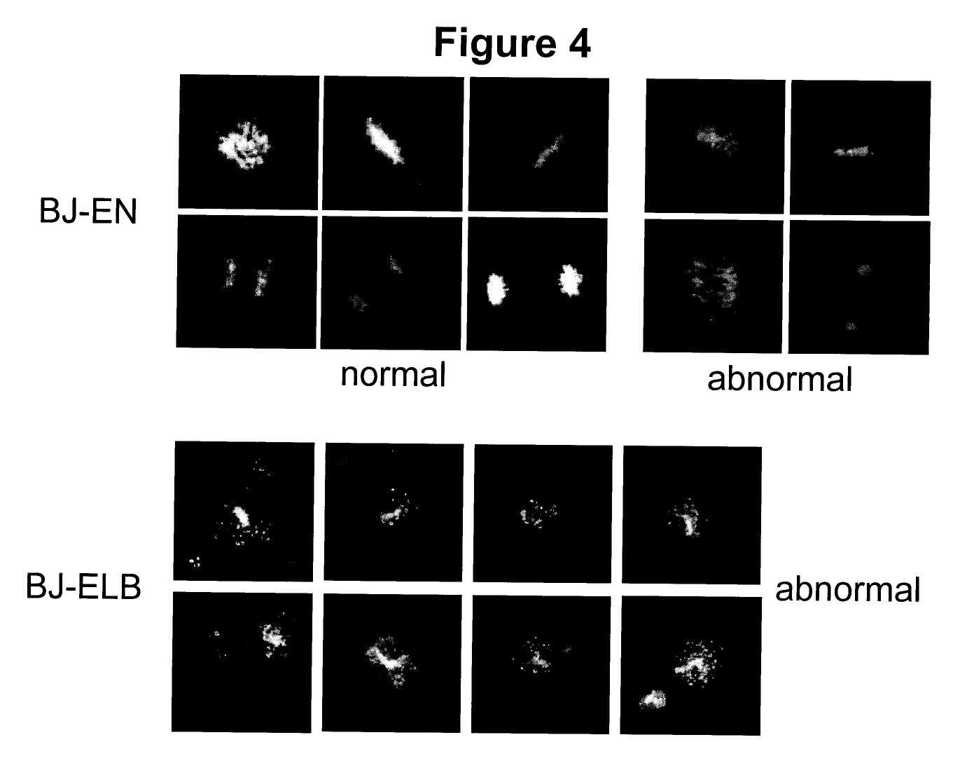

Alternatively, the cells can show strong increase in MI, reaching levels as high or higher than those of untreated cells. As shown herein (FIGS. 3A through 3E and 4), such an increase is characteristic of cells that undergo mitotic catastrophe, the duration of which is greatly extended relative to normal mitosis. In this case, the cells are assayed for mitotic catastrophe, for example, by microscopic examination of the cells to detect abnormal mitotic figures or micronuclei, or using any appropriate assay for mitotic catastrophe as set forth by illustration herein.

This screening strategy has several useful aspects, which, individually or in combination, distinguish it from all other cell-based assays for anticancer agents. These include: (i) reliance on changes in MI rather than in the cell number distinguishes cell cycle perturbation from non-specific growth inhibition; (ii) previous MI-based screening strategies were aimed at detecting an increase in MI (produced by agents that act directly at mitosis), whereas the primary screening criterion of the methods of the invention is a decrease in MI, produced by agents that arrest cells in interphase; (iii) Step 2 of the strategy embodied in the methods of the invention is based on changes in MI that occur after release from the inducing compound, rather than in the presence of the compound as used in earlier assays; (iv) to discriminate between mitotic catastrophe and apoptosis, screening is preferably carried out with tumor cells that have a limited apoptotic response, and the primary assays are carried out using the assays for mitotic rather than apoptotic cells.

The results set forth in the Examples below demonstrate that mitotic catastrophe (and its consequent apoptosis), but not senescence, is induced in transformed cells preferentially to normal cells after treatment with a commonly used, clinically useful anticancer agent (doxorubicin). Increased mitotic catastrophe in transformed cells was associated not only with a higher rate of mitosis after drug treatment but also with a higher frequency of abnormal (relative to normal) mitoses. These findings confirmed that the ability to induce mitotic catastrophe provides a basis for tumor cell specificity of a clinically useful anticancer agent. The ability to induce mitotic catastrophe in tumor cells can thus be used to identify tumor-specific cytotoxic compounds that are likely to be useful as anticancer drugs. Methods for screening agents that induce mitotic catastrophe are thus provided by the present invention.

In certain embodiments, the methods of the invention comprise the following steps:

1. Tumor cells are plated in multi-well plates and exposed to test compounds for a period of time sufficient to induce growth arrest (if the compounds are capable of growth inhibition), e.g. 24 hrs.

2. Plates are stained with a mitosis-specific antibody, such as MPM2, TG3 or GF7, and antibody binding is detected, for example by indirect immunofluorescence labeling, advantageously using a fluorescence plate reader. Compounds that decrease MI according to this assay are identified and used for further screening in step 3. Compounds that increase MI according to this assay are also identified and used for further screening in step 5.

3. Following treatment with the compounds that are identified in step 2 as decreasing MI, cells are allowed to recover for period(s) of time sufficient to allow compound-inhibited cells to re-enter the cell cycle (typically, 24 hrs, 36 hrs, and 48 hrs)

4. Plates from step 3 are used to measure MI as described in step 2. Compounds that produce an increase in MI similar to or higher than in untreated cells grown to the same density are identified as potential inducers of mitotic catastrophe. Compounds that produce no increase in MI or a weak increase (less than MI of untreated cells grown to the same density) are also identified as potential inducers of senescence.

5. Compounds identified in step 2 or step 4 by an increase in MI are added to cells, and mitotic figure morphology (during and after treatment with the compound) and whether micronuclei are present is analyzed by microscopic assays.

6. Compounds identified in step 4 by sustained decrease in MI are added to cells for 1-5 days, and tested for the expression of senescence markers (such as SA-β-gal) or the ability to abrogate long-term colony formation.

The above-described assays are useful for identifying compounds that will induce mitotic catastrophe or senescence in tumor cells.

Detection of Mitotic Catastrophe

The most common method for detecting mitotic catastrophe is based on scoring cells with fragmented nuclei. Such scoring can be done on unfixed cells (using phase contrast microscopy), or by bright-field microscopy after staining cells with any convenient dye that differentially stains nuclei (e.g. hematoxylineosin), or after DNA specific staining, using colored dyes such as Foelgen (for bright-field microscopy) or fluorescent dyes such as DAPI or Hoechst 33342 (for fluorescence microscopy). In identifying micronucleated cells as end points of mitotic catastrophe, it is important to distinguish them from apoptotic cells (which may result either from mitotic catastrophe or from mitosis-independent apoptosis). While apoptotic cells also have fragmented nuclei, they can be distinguished by small size and shrunken cytoplasm, whereas micronucleated cells are large and have normal-size cytoplasm. Furthermore, staining with DNA-specific dyes shows that apoptotic cells have condensed chromatin, whereas micronucleated cells are interphase cells having decondensed chromatin that arise after abnormal mitosis. Micronucleated cells may have two or more completely or partially separated nuclei; in the case of partial separation, the nuclei appear multilobulated. Representative examples of abnormal nuclear morphology that results from mitotic catastrophe (in HT1080 fibrosarcoma cells) are shown in FIG. 2. Another method for detecting micronuclei relies on the use of fluorescence-activated cell sorting (FACS), as described for example in Torres and Horwitz (1998, Cancer Res. 58: 3620-3626).

The morphological range of normal mitoses in a given cell line is first established by examination of mitotic figures in untreated cells, and deviations from normal morphology at any phase of mitosis can then be readily identified. Whereas micronucleation represents an end point of mitotic catastrophe, the process of abnormal mitosis can also be readily identified by microscopic analysis of cells stained with a DNA-specific detection reagent such as a dye (for example, DAPI) using standard procedures (see, for example, Freshney, 2000, Id.). Preferred procedures also include cells transfected with an expression vector for histone H2B-GFP fusion protein, which permits visualization of mitotic figures by fluorescence microscopy of intact cells, without any fixation or staining procedures (as disclosed in Kanda et al., 1998, Curr Biol 8: 377-385). Exemplarily, for this analysis, cells are cultured in media free of phenol red that provides some background fluorescence. Cells are examined using an inverted fluorescence microscope and mitotic figures photographed, to collect a sufficient number (typically, about 100) of mitotic images per sample. These mitotic figures are examined and classified with regard to the type of normal or abnormal mitoses that they represent, using the classification of mitotic figures in Therman and Kuhn (1989, Crit Rev. Oncog.1: 293-305); examples of abnormal mitotic figures (in DAPI-stained HT1080 fibrosarcoma cells) are shown in FIG. 2. Abnormal spindle formation or centrosome duplication can also be detected by staining with antibodies against α, βor γ tubulin. Another indication of abnormal mitosis is altered frequency distribution of different phases of mitosis. Characteristically, drug-induced abnormal mitoses are characterized by a lower frequency of anaphases and telophases, as well as abnormal morphology.

Time-lapse video microscopy (phase-contrast, DIC or fluorescence) can be used to establish the nature of abnormal mitosis induced by a tested compound. In a particular example of this type of analysis, fluorescence video microscopy of HT1080 cells expressing histone H2B-GFP fusion protein can be used (as illustrated in an online supplement to the Science review of Rieder and Khodjakov, 2003, Science 300: 91-96). For such analyses, H2B-GFP-expressing cells are advantageously plated onto 1″-diameter round glass cover slips and placed into wells of a 6-well plate. Media containing the test compound (in 1.5-mL volume) is added for 24 hrs, and then replaced with drug-free media. Plates are periodically examined for the reappearance of mitotic figures. Once mitoses begin to appear, the cover slip is transferred into a chamber of the incubator system for use with an inverted fluorescence microscope equipped with a heated stage. The chamber is filled with media containing HEPES, sealed airtight, and placed on the 37° C.-heated stage (or in a 37° C. thermal room, as needed). The microscope is connected to a digital time-lapse camera synchronized with an automatic shutter that allows fluorescent illumination only at the time of taking images. The images are collected intermittently, for example, using a 3-minute periodicity. A cell in early prophase is selected for filming, and it is monitored until the nuclear envelope(s) have been formed. From a single chamber, the duration of 2-5 mitoses are recorded. At least 20 mitoses are filmed for each promising hit and categorized. This analysis demonstrates which type(s) of abnormal mitosis are preferentially induced by the tested compound.

High-Throughput Screening for Mitotic Catastrophe or Senescence.

While microscopic analysis is not difficult, it is a relatively slow procedure for high-throughput screening (HTS). An approach to HTS for mitotic catastrophe is a simple and easily scalable procedure that can be used prior to microscopic examination, so that only compounds found to be positive in this preliminary screening need to be tested through microscopic assays. This preliminary step can be carried out as the primary screening assay or it can be used only with growth-inhibitory compounds, following preliminary screening for growth inhibitory activity (through conventional cell growth inhibition assays). The proposed screening procedure is schematized in FIG. 1 and it can also be used to screen for compounds that induce senescence in tumor cells.

With regard to induction of mitotic catastrophe, all anticancer drugs can be divided into two types. The first type comprises those drugs that directly affect mitosis and induce mitotic delay in tumor cells. This category includes anti-microtubular agents, such as Vinca alkaloids or taxanes; HDAC-I may also belong to this category. Mitotic index is increased in the presence of drugs of the first type, making an increase in MI in the presence of the drug a means of classifying these compounds. MI can be measured not only through microscopic counting but also much more conveniently, by staining with antibodies that specifically bind to mitotic cells, such as MPM2, TG-3 or GF-7 (Rumble et al., 2001, J Biol Chem. 276: 48231-48236). Increased binding of a mitosis-specific antibody (after exposure to ionizing radiation) has been used in the art as the basis for HTS of compounds that abrogate G2 checkpoint (Roberge et al., 1998, Id.; Rumble et al., 2001, Id.).

Most clinically-useful anticancer drugs (including doxorubicin) belong to the second type. These drugs induce cell cycle arrest in cell cycle interphase (i.e., in G1, S or G2), so that the MI decreases rather than increases in the presence of these drugs. MI, however, increases upon the removal of such drugs, as drug-inhibited cells reenter the cycle and proceed into mitosis (see FIG. 3E). This increase should be especially pronounced for drugs that induce mitotic catastrophe, since abnormal mitosis takes more time than normal mitosis. The increase in MI after removal of the drug can therefore indicate that cells recovering after drug treatment undergo mitotic catastrophe. On the other hand, the failure to increase MI to the level observed in untreated cells grown to the same density can indicate that some of the treated cells undergo prolonged growth arrest, which can be a consequence of senescence. The induction of either mitotic catastrophe or senescence by compounds identified by this screening procedure can then be verified through specific assays.

Measurement of MI.

The most common laboratory procedure for measuring MI is microscopic counting of cells with condensed chromatin, visualized by staining with DNA-specific dyes such as DAPI. While counting is a laborious and time-consuming procedure, it can be facilitated and automated using new microscopic techniques, such as laser-scanning microscopy. Prior art screening techniques based on MI measurement have relied on the binding of mitotic cell specific antibodies (MCSA), such as commercially available MPM2 or TG3 (Anderson et al., 1998, Exp. Cell Res. 238: 498-502). Notably, the MPM2 antibody was reported to stain only mitotic but not apoptotic cells (Yoshida et al., 1997, Exp. Cell Res. 232: 225-239). MCSA have been used in the published screening assays for an increase in MI through either cytoblot (Haggarty et al., 2000, Id.) or modified ELISA procedures (Roberge et al., 1998, Id.; Roberge et al., 2000, Id.). Another method for MCSA-based measurement of mitotic cells relies on the use of FACS, which provides a quantitative measurement of the fraction of MCSA-binding cells (which is a good approximation of MI). FACS assays are also advantageous because they permit determination of not only MI but also the total number of cells in the sample. Furthermore, FACS assays allow one to combine MCSA staining with propidium iodide (PI) staining for DNA content, making it possible to combine the measurement of MI with G1 or G2 growth arrest and with the appearance of apoptotic cells having sub-G1 DNA content. Recent advances in FACS instrumentation, in particular the development of an automatic FACS Multiwell AutoSampler (Becton Dickinson) make it possible to use FACS as a rapid screening procedure, which is preferred in the practice of the methods of the present invention.

Cells and Compound Libraries.

In principle, any cell line can be used for screening, but a tumor-derived cell line is preferred, since the ultimate goal of the screening procedure is to identify new drugs effective against tumor cells. Particularly preferred tumor cell lines are those that have a low incidence of apoptosis, since rapid onset of apoptosis may obscure the detection of senescent cells or cells undergoing mitotic catastrophe. Apoptosis-resistant lines can be selected among the lines that are intrinsically resistant to apoptosis or that were rendered apoptosis resistant by overexpression of an apoptosis-inhibiting gene, such as BCL2. An example of a convenient cell line for the practice of the methods of the invention is HT1080 human fibrosarcoma, which has only very low incidence of apoptosis (Pellegata et al., 1996, Proc. Natl. Acad. Sci. U.S.A. 93: 15209-15214; Chang et al., 1999, Cancer Res. 59: 3761-3767; co-owned and co-pending U.S. Ser. No. 09/958,457, filed Apr. 7, 2000, incorporated by reference herein).

Screening can be carried out with any of a number of commercially-available or custom-made libraries of natural or synthetic compounds. An example of a commercially available library is ChemBridge DIVERSet, a sub-set of ChemBridge collection of synthetic compounds, rationally chosen by quantifying pharmacophores in the entire collection, using a version of Chem-X software. The resulting library provides the maximum pharmacophore diversity within the minimum number of compounds. This library has been successfully used by many industrial and academic researchers, in a variety of cell-based and cell-free assays (www.chembridge.com). In particular, the ChemBridge library has been used to identify monastrol that interferes with mitosis by inhibiting mitotic spindle bipolarity (Mayer et al., 1999, Id.) and many other inhibitors of mitosis identified by screening for their ability to increase MI (Haggarty et al., 2000, Id.). In the latter study, 16,320 compounds from the ChemBridge library were screened, and 139 compounds were found to increase MI. These results promote confidence that using the same library a large number of compounds that inhibit the cell cycle with subsequent effects on mitosis can be found. The most current ChemBridge DIVERSet library contains 30,000 compounds in 5 μmol samples, pre-plated and dissolved in 500 μL DMSO. According to the methods of this invention, as disclosed more fully herein, screening assays are carried out at 20 μM concentration of each compound (typically used in the art for cell-based assays); thus the total amount of each compound in the library is sufficient to prepare 250 mL of media. This is more than sufficient for all screening purposes. For larger-scale analysis, individual hits can be re-supplied by ChemBridge in 10 mg vials.

Assay Optimization.

In preparation for screening, the most suitable multiwell plates for the assay and the densities at which cells can be grown in such plates are identified. Initial optimization of the assays useful in the practice of the methods of this invention are carried out using untreated cells, to determine well-to-well variability and the range of MI values in different experiments. These optimization assays demonstrate that the assay works in a 96-well format or in a 24-well format. When the first-step assay conditions are established with untreated cells, the ability to detect cell cycle inhibitors is tested using several known drugs with different cell cycle specificity. These can include taxol (that arrests cells in mitosis and therefore increases MI), and several drugs that arrest cells in the interphase, decrease MI, and induce mitotic catastrophe and/or senescence. The latter agents can include mimosine (arrest at G1/S boundary), aphidicolin (S-phase arrest) and doxorubicin (late S and G2 arrest). The dose range for inhibiting HT1080 cell growth with these compounds has been established (Levenson et al., 2000, Cancer Res. 60: 5027-5030). Lovastatin, reported to inhibit some tumor cell lines in G1 (Keyomarsi et al., 1991, Cancer Res 51: 3602-3609), is another candidate for testing whether it inhibits tumor cell growth with HT1080 cells and whether it induces mitotic catastrophe.

Advantageously cells are treated with several doses of each drug (covering the range from LD₅₀ to LD₉₉) in the 96-well assay format (in triplicates), and the effects of 24-hr incubation on MI are established by FACS assay. The lowest dose of each compound that produces at least 2-fold decrease in MI (or 5-10 fold increase in MI in the case of taxol) is selected, and the reproducibility of the effect of each compound on MI is tested, by adding the drug to multiple wells at different positions in the plate. This analysis verifies the reproducibility of the assay, provides the range of variability for the effects of the same drug, and reveals potential position-related problems in the assay. Established doses of one or more of these drugs are used as positive controls for the actual screening of compound library.

Whereas the decrease in MI constitutes a preferred identifier for interphase-active drugs, it may be advantageous in some cases to use an alternative assay in the first step, wherein cells are incubated with the tested compound and then with a known anti-mitotic agent such as nocodazole (for eight hours or a similar period of time; Roberge et al., 1998, Id.). A compound that inhibits interphase should interfere with nocodazole-mediated increase in MI. Disadvantageously, as compared to the MI-decrease assay, this nocodazole assay is longer and requires the use of an additional drug; it is also unsuitable for identifying compounds that increase rather than decrease the MI. Nevertheless, the nocodazole assay has a potential advantage of increasing the measured signal (i.e., MI) and may therefore allow one to use fewer cells for FACS analysis (or cytoblot or ELISA assays).

The same prototype drugs are also used to establish the conditions for the second step of the screening procedure. This analysis can require up to 3-5 days of cell culture, and is preferably carried out in the 24-well format. Typically, drugs are added to the cells for 24 hrs and then replaced with drug-free media. Multiwell plates are fixed and processed at different time points after release from the drug (6-72 hrs, with 6-hr intervals for the first 24 hrs and 8-hr intervals for the next 48 hrs), and FACS analysis used to determine the MI. This analysis reveals the timing and the magnitude of the recovery of MI in cells released from drugs that arrest cell cycle in different phases, as well as the number of cells remaining at different times after release. Based on this analysis, two (or, if necessary, three) time points are selected that correspond to the reentry into mitosis by cells treated with different drugs. The reproducibility of this release assay is determined essentially as in the first step. The results of this analysis provide the time parameters for the second step of screening and with positive controls for the second step of screening.

Morphological Assays for Mitotic Catastrophe and Senescence.

To determine whether compounds that decrease MI when cells are incubated in their presence but produce an increase in MI after release actually induce mitotic catastrophe, such compounds are tested by morphological analysis of the mitotic figures, as described above.

To determine if compounds that decrease MI and do not permit full recovery after release induce senescence, cells treated with such a compound for two or more days are stained for senescence-associated β-galactosidase (SA-β-gal) activity, using X-Gal at pH6.0, as described by Dimri et al. (1995, Proc. Natl. Acad. Sci. USA 92: 9363-9367). Blue staining (detectable by light microscopy) indicates expression of this commonly-used marker of senescence. In addition, senescent cells show increased cell size and higher granularity (as evidenced by increased side scatter in FACS analysis). As a functional test for senescence, cells are treated with the compound or untreated and then plated at a low density (500-2000 cells per P100) and allowed to form colonies. Senescent cells show greatly decreased formation of large colonies relative to untreated cells, but microscopic observation indicates that most of the plated cells remain attached to the plate, while remaining as single cells or forming very small clusters.

Further Characterization of the Screened Compounds

The procedures described above are used to identify compounds that induce either mitotic catastrophe or senescence. The most effective compounds are then advantageously further characterized as potential anticancer drugs by more conventional in vitro assays, such as dose response analysis using short-term growth inhibition and long-term clonogenic assays, to establish the ID₅₀ and LD₅₀ values for comparison with other drugs. The spectrum of activity of the compounds is profiled in different human tumor cell lines, and in particular in unmodified or telomerase-immortalized normal cells (as described in Example 1) below, to determine if the compound is likely to have a tumor-specific effect. The most promising compounds can be derivatized by conventional techniques, and the derivatives can be screened again for the induction of senescence or mitotic catastrophe. Subsequent in vivo testing can determine the efficacy of the compounds in animal models of cancer, such as xenografts of human tumors grown in immunodeficient mice, or transgenic mouse models of specific cancers. Conventional animal tests are also used to determine the safety and bioavailability of the compounds, in preparation for clinical studies that would validate such compounds as anticancer drugs.

The methods of the invention are useful for identifying compounds that inhibit the growth of tumor cells, most preferably human tumor cells. The invention also provides the identified compounds and methods for using the identified compounds to inhibit tumor cell, most preferably human tumor cell growth.

The invention also provides embodiments of the compounds identified by the methods disclosed herein as pharmaceutical compositions. The pharmaceutical compositions of the present invention can be manufactured in a manner that is itself known, e.g., by means of a conventional mixing, dissolving, granulating, dragee-making, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries that facilitate processing of the active compounds into preparations that can be used pharmaceutically. Proper formulation is dependent upon the route of administration chosen.

Non-toxic pharmaceutical salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—CH₃ where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

For injection, tumor cell growth-inhibiting compounds identified according to the methods of the invention can be formulated in appropriate aqueous solutions, such as physiologically compatible buffers such as Hank's solution, Ringer's solution, or physiological saline buffer. For transmucosal and transcutaneous administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, phosphoric, hydrobromic, sulfinic, formic, toluenesulfonic, methanesulfonic, nitric, benzoic, citric, tartaric, maleic, hydroiodic, alkanoic such as acetic, HOOC—(CH₂)ₙ—CH₃ where n is 0-4, and the like. Salts tend to be more soluble in aqueous or other protonic solvents that are the corresponding free base forms. Non-toxic pharmaceutical base addition salts include salts of bases such as sodium, potassium, calcium, ammonium, and the like. Those skilled in the art will recognize a wide variety of non-toxic pharmaceutically acceptable addition salts.

Pharmaceutical compositions of the compounds of the present invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, for example, via injection of the compound directly into a specific tissue, often in a depot or sustained release formulation.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays, as disclosed herein. For example, a dose can be formulated in animal models to achieve a circulating concentration range that includes the EC₅₀ (effective dose for 50% increase) as determined in cell culture, i.e., the concentration of the test compound which achieves a half-maximal inhibition of tumor cell growth. Such information can be used to more accurately determine useful doses in humans.

It will be understood, however, that the specific dose level for any particular patient will depend upon a variety of factors including the activity of the specific compound employed, the age, body weight, general health, sex, diet, time of administration, route of administration, and rate of excretion, drug combination, the severity of the particular disease undergoing therapy and the judgment of the prescribing physician.

Preferred compounds of the invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcová et al. (1996, J. Chromat. B 677: 1-27). Compound half-life is inversely proportional to the frequency of dosage of a compound. In vitro half-lives of compounds may be predicted from assays of microsomal half-life as described by Kuhnz and Gieschen (1998, DRUG METABOLISM AND DISPOSITION, Vol. 26, pp. 1120-1127).

Toxicity and therapeutic efficacy of such compounds can be determined by standard pharmaceutical procedures in cell cultures or experimental animals, e.g., for determining the LD₅₀ (the dose lethal to 50% of the population) and the ED50 (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch.1, p.1).

Dosage amount and interval can be adjusted individually to provide plasma levels of the active moiety that are sufficient to maintain tumor cell growth-inhibitory effects. Usual patient dosages for systemic administration range from 100-2000 mg/day. Stated in terms of patient body surface areas, usual dosages range from 50-910 mg/m²/day. Usual average plasma levels should be maintained within 0.1-1000 μM. In cases of local administration or selective uptake, the effective local concentration of the compound cannot be related to plasma concentration.

The following Examples are intended to further illustrate certain preferred embodiments of the invention and are not limiting in nature.

### EXAMPLES

### Example 1

### Doxorubicin Preferentially Induces Mitotic Catastrophe in Neoplastically Transformed Fibroblasts

Doxorubicin, a commonly used drug with proven clinical utility in the treatment of different cancers, was chosen as an exemplary chemotherapeutic agent to demonstrate the efficacy of the methods of the invention for identifying agents that kill checkpoint-deficient human cells preferentially to normal cells.

An isogenic pair of telomerase-immortalized human fibroblasts was used in these assays. One of the pair of human fibroblasts was transduced by the early region of SV40, resulting in checkpoint control debilitation and partial transformation. These cell lines were derived from BJ primary human fibroblasts (Accession No. CRL-2522, American Type Culture Collection, Manassas, Va.) after retroviral transduction with the human telomerase protein component (hTERT), or with a combination of hTERT with the early region of SV40 that encodes large-T (LT) and small-T (ST) oncogenes (Hahn et al., 1999, Nature 29: 464-468; Hahn et al., 2002, Mol. Cell. Biol. 22: 2111-2123). The cell line transduced with hTERT alone was designated BJ-EN, and the line transduced with hTERT and early region of SV40 was called BJ-ELB. Both the BJ-EN and BJ-ELB cell lines were provided by Dr. William Hahn (Massachusetts General Hospital, Boston, Mass.). These cell lines were cultured in a 4:1 mixture of DMEM and Medium 199, with 10% fetal calf serum, supplemented with glutamine, pyruvate, penicillin and streptomycin.

hTERT-transduced BJ fibroblasts are immortal, but they maintain all the other properties of normal (untransformed) cells, including normal karyotype, contact inhibition, and inability to grow in soft agar or form tumors in animals, and the ability to undergo senescence in response to mutant RAS (Jiang et al., 1999, Nat. Genet. 21: 111-114; Hahn et al., 1999, Id.). Introduction of the SV40 early region encoding LT and ST results in a partially-transformed phenotype (Hahn et al., 2002, Id.). LT disables the retinoblastoma and p53 tumor suppressor pathways, thus disabling most of the cellular checkpoint controls. ST perturbs protein phosphatase 2A, which results in the stimulation of cell proliferation and anchorage-independent growth (Hahn et al., 2002, Id.).

The growth rate of BJ-EN and BJ-ELB cell lines was compared in the absence of a drug, by plating cells in 6-well plates, at a concentration of 25,000 cells per well, and determining cell numbers on consequent days using a Coulter counter. As shown in FIG. 3A, the untransformed BJ-EN cells grow much more slowly than the partially transformed BJ-ELB cells. The effects of 3-day exposure to different concentrations of doxorubicin on cell growth in these cell lines was then determined. As shown in FIG. 3B, the untransformed BJ-EN cells were more resistant to doxorubicin than BJ-ELB cells (except for the lowest drug doses), indicating that doxorubicin shows a transformed-cell specificity in this system. For comparative analysis of specific cellular responses, a concentration of 30 nM doxorubicin was chosen, which had approximately equal growth-inhibitory effect in both cell lines (FIG. 3B).

In the comparative assays, equal numbers of cells were plated, and the following day doxorubicin was added to a final concentration of 30 nM. Cells were cultured with doxorubicin at this concentration for 3 days and then transferred into drug-free media for three more days. FIG. 4C shows changes in the absolute cell numbers over the course of this experiment. The untransformed BJ-EN cells showed essentially no change in cell number during doxorubicin treatment, indicating a cytostatic effect of the drug on the immortalized but cell-cycle unperturbed cells; BJ-EN cell number did not change significantly over three days after release from the drug. In contrast, BJ-ELB cells increased their number on the first day of doxorubicin, indicating inefficient cell cycle arrest resulting in continued growth, but by day 3 after release (3 dR) the cell number in this cell line eventually decreased to the same value as at the time of doxorubicin addition (d0), suggesting cell death (FIG. 3C).

For morphological evaluation of different cellular responses to doxorubicin, one aliquot of cells at each time point was stained for the senescence marker SA-β-gal as described in Dinri et al. (1985, Id.), and another aliquot was fixed with methanol/acetic acid and stained with DAPI (a DNA-specific fluorescent dye) for fluorescence microscopy analysis. The percentage of senescent (SA-β-gal positive) cells showed a similar increase in both cell lines (FIG. 3D), indicating that transformation-associated changes produced by SV40 early region did not significantly alter the senescence response to drug treatment.

On the other hand, analysis of DAPI-stained cells showed a great increase in the fraction of cells with multiple micronuclei in partially transformed BJ-ELB relative to the untransformed BJ-EN cells, indicative of mitotic catastrophe. Two days after release, the micronucleated cell fraction was 1.7% in BJ-EN cells but 26.4% in BJ-ELB cells. The fraction of cells with apoptotic morphology (shrunken cells with condensed and broken chromatin) was not as high as the fraction of micronucleated cells, but it was also higher in BJ-ELB (6.6%) than in BJ-EN (0.9%). As indicated above, apoptosis in doxorubicin-treated cells is also likely to be a consequence of mitotic catastrophe. Thus, doxorubicin-induced mitotic catastrophe is much less common in normal cells than in checkpoint-deficient transformed cells.

To identify the causes of increased mitotic catastrophe in transformed cell lines, fluorescence microscopy of DAPI-stained cells was used to determine the percentage of mitotic cells (mitotic index, MI) at different points of the experiment. As shown in FIG. 3E, the addition of doxorubicin resulted in immediate and complete cessation of mitosis in the normal BJ-EN cells. In contrast, mitosis was only partially inhibited in BJ-ELB cells. The MI values drastically increased in BJ-ELB cells on the second day after release (sharply decreasing on the following day), but the resumption of mitosis was much less pronounced in BJ-EN cells (FIG. 3E). In particular, on day 2 after release from the drug, the MI of BJ-EN cells was only 0.4%, but the MI of BJ-ELB rose to 8.0%.

The above results indicated that the higher rate of mitotic catastrophe in partially transformed cells results at least in part from a higher fraction of such cells entering mitosis during and after doxorubicin treatment. An additional reason for increased mitotic catastrophe could be a difference in the “quality control” of mitosis between BJ-EN and BJ-ELB cells that enter mitosis after release from the drug. To resolve this issue, fluorescence microscopy of DAPI-stained cells was used to compare the ratio of normal and abnormal mitotic figures in these cell lines two days after release from doxorubicin. The untransformed BJ-EN cells showed 60% normal and 40% abnormal mitotic figures, whereas only 8% of mitotic figures in BJ-ELB cell line appeared normal. Examples of mitotic figures of the two cell lines are provided in FIG. 4. Characteristically, 29% of mitotic figures in BJ-EN cells were metaphases and telophases, whereas only 1% of mitotic figures in BJ-ELB cell line represented anaphase or telophase. Hence, the partially transformed and untransformed cell lines differed not only in the rate but also in the quality of mitosis after release from doxorubicin.

Thus, mitotic catastrophe (and its consequent apoptosis), but not senescence, is induced in transformed cells preferentially to normal cells after doxorubicin treatment. These results provide a direct demonstration that a clinically useful anticancer agent (doxorubicin) induces mitotic catastrophe in transformed cells preferentially to normal cells. Screening compounds for the ability to induce mitotic catastrophe in tumor cells is therefore a useful approach to the identification of new anticancer drugs.

### Example 2

### Determination of Mitotic Index and Demonstration of Mitotic Catastrophe

Mitotic index and the incidence of mitotic catastrophe were determined using mitotic cell specific antibodies (MCSA) as follows. Three different MCSA were compared using fluorescence activated cell sorting (FACS) based on immunofluorescence labeling with MCSA coupled with propidium iodide (PI) staining for DNA content. In this procedure, cells were washed, trypsinized, fixed with an equal volume of 70% ethanol (on ice), resuspended in a small volume of 1% BSA-PBS containing an MCSA, incubated for 1 hour at room temperature, and then washed and bound with secondary (fluorescently-labeled) antibody. The tested MCSA included MPM2 (available from Upstate Biotechnology, Cat. #05-368) and two antibodies provided by Dr. P. Davies (Albert Einstein College of Medicine), including the previously characterized TG3 (Anderson et al., 1998, Exp. Cell Res. 238: 498-502) and unpublished GF7 antibody. The fractions of exponentially growing (untreated) HT1080 cells that bound the antibody and had G2/M DNA content were 2.42±0.29% for GF7, 2.27±0.71% for MPM2 and 1.76±0.57% for TG3.

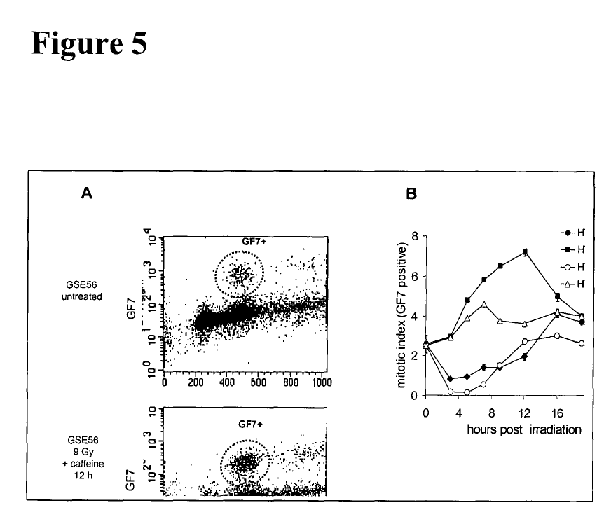

The utility of MCSA for detecting both an increase and a decrease in MI is illustrated by the experiment in FIGS. 5A and 5B. FACS analysis of GF7/PI stained cells was used to analyze radiation-induced changes in the MI of HT1080 fibrosarcoma cells with different cell cycle checkpoint integrity status. The following cells were used in these assays: wild-type HT1080 cells, which have functional G1 and G2 checkpoints; HT1080 cells transduced with GSE56, a genetic inhibitor of p53 that abrogates the G1 checkpoint and weakens the G2 checkpoint; and cells treated with 4 mM caffeine, which abrogates the G2 checkpoint. Representative staining of untreated and irradiated cells is shown in FIG. 5A. The time course of changes in MI of irradiated HT1080 cells, in the presence and in the absence of GSE56 or caffeine, is shown in FIG. 2B (each point in FIG. 5B represents triplicate assays). Shortly after irradiation in the absence of caffeine, wild type HT1080 cells showed a temporary decrease in MI almost to zero, reflecting G2 checkpoint activation. GSE56-transduced cells also showed a drop in MI, albeit not as complete as in the wild-type cells, due to the effects on the G2 checkpoint of the GSE. In the presence of caffeine, however, MI did not decrease but rather increased nearly 2-fold in the wild-type HT1080 cells and up to 3-fold in GSE56-transduced cells. These results showed that MCSA-based FACS measurement of MI was a sensitive technique for measuring either an increase or a decrease in MI in cells treated under different conditions.

To simplify the screening procedure, instead of using the secondary antibody, MCSA can be labeled directly using, for example, the Zenon kit from Molecular Probes (http://www.probes.com/products/zenon/). Zenon technology is based on complexing primary antibodies with dye- or enzyme-labeled Fab fragments of secondary antibodies directed against the Fc regions of the primary antibody. Zenon labeling conditions are optimized for MCSA as described in Zenon protocols, and Zenon Fab fragments conjugated with different fluorescent dyes are tested and compared for optimal detection. For screening, cells are grown in Millipore MultiScreen 96-well filter plates with detachable trays (such as MultiScreen-FL), where cells can be consecutively incubated with various solutions and rinsed in the same plates by vacuum filtration. MultiScreen-FL filter plates were shown to be suitable for similar immunostaining procedures, according to Millipore technical literature (http://www.millipore.com/publications.nsf/ docs/PS1005EN00). One of the advantages of the Multiscreen filter plates is that the initial collection of cells onto polycarbonate filters by vacuum filtration combines the attached and the floating cells, thus avoiding the loss of accidentally detached mitotic cells. Starting with the Millipore protocols, trypsinization, fixation, rinsing and antibody labeling procedures are optimized in this setup, and the minimal number and duration of steps necessary for immunofluorescence labeling are established. Alternatively, antibody staining and washing in the process of screening can be carried out using automated robotic systems, such as Zymark Cell Station

For FACS analysis, antibody-labeled cells are suspended in 50 μL PBS containing 100 μg/mL RNAse and 5 μg/mL PI and incubated for 15-30 minutes at 37° C.. The same plates are then placed into the Becton Dickinson (BD) Multiwell AutoSampler (50 μL is an adequate sample volume for the AutoSampler, according to BD). Cell suspensions are automatically loaded and analyzed in a FACS system, such as BD FACSCalibur. According to BD, the processing time for the 96-well plate for this system is 14 minutes at optimal cell concentrations. The data are recorded and analyzed using BD FACStation Data Management System. FACS analysis provides the total number of cells, the cell cycle distribution in the treated populations, the fraction of apoptotic (sub-GI) cells, and the fraction of MCSA+ cells with G2 DNA content (the measure of MI).

Using such assays, determination of mitotic index and detection of mitotic catastrophe can be used for rapid, high throughput screening of compounds to detect anticancer agents with specificity for tumor cells.

It should be understood that the foregoing disclosure emphasizes certain specific embodiments of the invention and that all modifications or alternatives equivalent thereto are within the spirit and scope of the invention as set forth in the appended claims. All references cited herein are incorporated by reference in their entirety.

We claim:

1. A method for identifying a compound that induces tumor-specific cell death, comprising the steps of
a) contacting a culture of cancer cells with a test compound;
b) detecting induction of mitotic catastrophe, by assessing mitotic figure morphology in the treated cells or detecting interphase cells with two or more micronuclei;
c) identifying compounds that induce mitotic catastrophe as inducers of tumor-specific cell death.

2. A method according to claim 1, wherein the test compound is removed from the culture after contacting the cells, and the cells are cultured in the absence of the test compound before detecting induction of mitotic catastrophe in step (b).

3. A method according to claim 1, wherein the compound identified in step (c) is contacted with non-cancer cells and assayed for induction of cell death in said non-cancer cells, wherein compounds that do not induce cell death in said non-cancer cells, or that induce cell death in said non-cancer cells to a lesser extent or degree than in cancer cells, are identified.

4. A method for identifying a compound that induces mitotic catastrophe or senescence in cancer cells, comprising the steps of:
a) contacting a culture of cancer cells with the compound for a time and at a compound concentration sufficient to induce cell growth arrest;
b) assaying a portion of the treated cells to detect a decrease in the mitotic index of the treated cells;
c) removing the compound and culturing the cells for a recovery period for a time sufficient to permit the cells to re-enter the cell cycle;
d) assaying a portion of the recovered cells to detect an increase in the mitotic index of the recovered cells;
e) assaying compounds producing mitotic index smaller than in untreated cells for induction of senescence, by detecting production of senescence markers or long-term growth arrest in said cells;
f) assaying compounds producing mitotic index as large or larger than in untreated cells for mitotic catastrophe, by assessing the morphology of mitotic figures in the treated and recovered cells or by detecting the appearance of interphase cells with two or more micronuclei; and
g) identifying compounds that induce small increases in mitotic index and senescence markers or long-term growth arrest as senescence-inducing compounds in cancer cells, and identifying compounds that induce abnormal mitotic figures or micronuclei in the cells as compounds that induce mitotic catastrophe in cancer cells.

5. The method of claim 4, wherein the cells are human cancer cells.

6. The method of claim 4, wherein the cells are solid tumor cells.

7. The method of claim 4, wherein the cells are HT1080 cells.

8. The method of claim 4, wherein the cells are contacted with the compound in step (a) for at least 3 hours.

9. The method of claim 4, wherein the cells are assayed in step (b) to detect a decrease in the mitotic index by staining a portion of the treated cells with a mitosis-specific reagent.

10. The method of claim 9, wherein the mitosis-specific reagent is a mitotic cell-specific antibody.

11. The method of claim 9, wherein the cells are assayed by microscopy.

12. The method of claim 9, wherein the cells are assayed by fluorescence-activated cell sorting.

13. The method of claim 4, wherein the cells are assayed in step (d) to detect an increase in the mitotic index by staining a portion of the recovered cells with a mitosis-specific reagent.

14. The method of claim 13, wherein the mitosis-specific reagent is a mitotic cell-specific antibody.

15. The method of claim 13, wherein the cells are assayed by microscopy.

16. The method of claim 13, wherein the cells are assayed by fluorescence-activated cell sorting.

17. The method of claim 4, wherein the senescence marker assayed in step (e) is senescence-associated beta-galactosidase (SA-β-gal).

18. The method of claim 4, wherein mitotic morphology is assayed using a DNA-specific detection reagent.

19. The method of claim 18, wherein the cells are assayed by microscopy.

20. The method of claim 4, wherein chromosomal morphology is assayed using an H2B-GFP fusion protein.

21. A method for inhibiting tumor cell growth, the method comprising the steps of contacting a tumor cell with an effective amount of a compound that induces mitotic catastrophe in a cancer cell, wherein the compound is identified according to the method of claims 1 or 4.

22. A method for treating a disease or condition relating to abnormal cell proliferation or tumor cell growth, the method comprising the steps of contacting a tumor cell with an effective amount of a compound that induces mitotic catastrophe in a cancer cell, wherein the compound is identified according to the method of claims 1 or 4.

23. A compound that that induces mitotic catastrophe in a cancer cell, wherein the compound is identified according to the method of claims 1 or 4.

24. A method for inducing senescence in a cancer cell, the method comprising the steps of contacting a tumor cell with an effective amount of a compound identified in step (g) of the method of claim 4.

25. A method for treating a disease or condition relating to abnormal cell proliferation or tumor cell growth, the method comprising the steps of contacting a tumor cell with an effective amount of a compound that induces senescence in a cancer cell, wherein the compound is identified in step (g) of the method of claim 4.

26. A compound that induces senescence in a cancer cell, wherein the compound is identified in step (g) of the method of claim 4.

27. A method according to claim 4, wherein the compound identified in step (g) as an inducer of mitotic catastrophe is contacted with non-cancer cells and assayed for cell death in said non-cancer cells, wherein compounds that do not induce cell death in said non-cancer cells, or that induce cell death in said non-cancer cells to a lesser extent or degree than in cancer cells, are identified.
